(19) United States
(12) Patent Application Publication
(10) Pub. No.: US 2006/0116430 A1
(43) Pub. Date: Jun. 1, 2006

(54) Method for the production of hydrocarbon liquids using a fischer-tropf method

(76) Inventors: Paul Wentink, Feucherolles (FR); Denis Cieutat, Neuilly S/Seine (FR); Guillaume De Souza, Issy les Moulineaux (FR)

Correspondence Address: AIR LIQUIDE, 2700 POST OAK BOULEVARD, SUITE 1800, HOUSTON, TX, 77056, US

(21) Appl. No.: 10/541,127

(22) PCT Filed: Apr. 2, 2004
(86) PCT No.: PCT/FR04/50141

§ 371 Date: Sep. 6, 2005

(30) Foreign Application Priority Data
Apr. 15, 2003 (FR) 0304698

(51) Int. Cl. C07C 27/26

(52) U.S. Cl. 518/726

(57) ABSTRACT

Methods for converting hydrocarbon gases into hydrocarbon liquids through Fischer-Tropsch methods. In addition to liquid hydrocarbons, a waste gas containing hydrogen, carbon dioxide, and hydrocarbons with less than 6 carbon atoms, is produced. The waste gas is separated and several gas streams are produced. One such gas stream contains methane, and has a recovery rate, in terms of hydrogen and carbon monoxide, of at least 60%. Another gas stream has a recovery rate, in terms of carbon dioxide, of at least 40%. A supplementary gas stream, which contains hydrocarbons with at least 2 carbon atoms, is also created.

The present invention relates to a novel method for converting gaseous hydrocarbons to liquid hydrocarbons using one of the known methods for generating synthesis gas, as well as the Fischer-Tropsch process and in particular, a specific step for treating the waste gas produced by the Fischer-Tropsch process.

It is well known how to convert raw gaseous or solid hydrocarbon compounds to liquid hydrocarbon products usable in the petrochemical industry, in refineries or in the transport sector. Some large natural gas fields are located in remote places and far from any consumer areas; they can accordingly be used by installing so-called “Gas to Liquid (GtL)” conversion plants near these natural gas sources. The conversion of the gases to liquids permits easier transport of the hydrocarbons. This type of GtL conversion is usually carried out by converting raw gaseous or solid hydrocarbon compounds to a synthesis gas mainly comprising H₂ and CO (by partial oxidation using an oxidizing gas and/or reaction with steam or CO₂), followed by the treatment of this synthesis gas by the Fischer-Tropsch process to obtain a product which, after condensation, yields the desired liquid hydrocarbon products. During this condensation, a waste gas is produced. This waste gas contains low molecular weight hydrocarbon products and unreacted gases. In consequence, it is generally used as a fuel in one of the processes of the GtL unit, for example in a gas turbine or a combustion chamber associated with a steam turbine or in an expansion turbine associated with a compressor of the GtL unit. However, the quantity of waste gas to be burned often substantially exceeds the fuel demand of the GtL unit. Moreover, the waste gas also comprises CO₂, which reduces the hydrocarbon product combustion efficiency and which is released into the atmosphere, in violation of environmental standards. Finally, the waste gas generally comprises amounts of unconverted H₂ and CO: hence it is not economical to bum them.

Considering the environmental constraints pertaining to CO₂, it has been proposed to treat the waste gas to strip it of CO₂. U.S. Pat. No. 5,621,155, for example, describes a method in which a portion of the waste gas from the Fischer-Tropsch process is treated in order to remove the carbon dioxide and is then recycled through the step of the Fischer-Tropsch process. However, the remaining portion of waste gas containing H₂ and CO is always burned, and this is uneconomical. Moreover, CO₂ is always released.

WO 01/60773 also describes a method in which the waste gas from the Fischer-Tropsch process is treated to strip it of CO₂. The waste gas with reduced CO₂ content is used as a fuel in various parts of the plant.

U.S. Pat. No. 6,306,917 describes a method in which the carbon dioxide is removed from the waste gas produced by the Fischer-Tropsch process. This patent also describes the treatment of the waste gas to recover the hydrogen using a membrane and the recycling of this hydrogen to the Fischer-Tropsch reactor. The CO compound is sent to combustion.

The object of the present invention is to propose a method for converting gaseous hydrocarbons to liquid hydrocarbons using the Fischer-Tropsch process in which the waste gas from this Fischer-Tropsch process is treated in order to avoid the economic loss of H₂ and CO by simple combustion.

A further object is to propose a method for converting gaseous hydrocarbons to liquid hydrocarbons using the Fischer-Tropsch process in which the waste gas is treated in order both to avoid the economic loss of H₂ and CO by simple combustion and to sharply reduce the atmospheric release of CO₂ by recycling the carbon chains.

The invention has the advantage of adapting to all types of waste gas. Moreover, it allows the re-use, in the GtL process, of the hydrocarbons present in the waste gas. The invention has the major advantage of performing the function of redistributing the various compounds of the waste gas in a plurality of gas streams usable in different steps of the general method for converting gaseous hydrocarbons to liquid hydrocarbons.

For this purpose, the invention relates to a method for converting gaseous hydrocarbons to liquid hydrocarbons in which the Fischer-Tropsch process is employed, said process producing liquid hydrocarbons and a waste gas comprising at least hydrogen, carbon monoxide, carbon dioxide and hydrocarbons with a maximum of 6 carbon atoms, and in which the waste gas is subjected to a separation method producing:
at least one gas stream comprising methane and for which the recovery rate of hydrogen and carbon monoxide is at least 60%,
at least one gas stream for which the carbon dioxide recovery rate is at least 40%, and
at least one supplementary gas stream mainly comprising hydrocarbons with at least 2 carbon atoms.

Other features and advantages of the invention will appear from a reading of the following description. Embodiments of the invention are given by way of non-limiting examples, illustrated by the drawings appended hereto, in which:

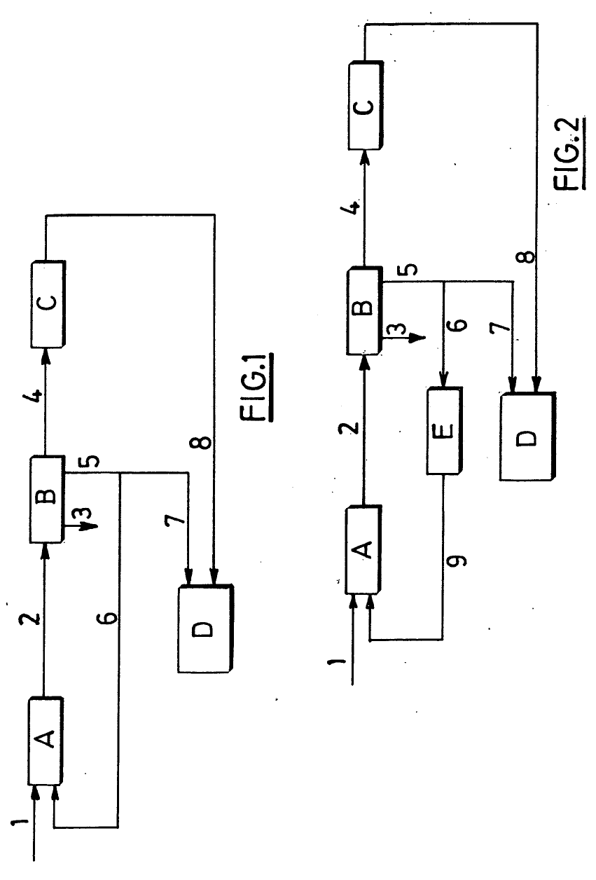

FIGS. 1 and 2 are flowcharts of a GtL unit incorporating a Fischer-Tropsch process according to the prior art,

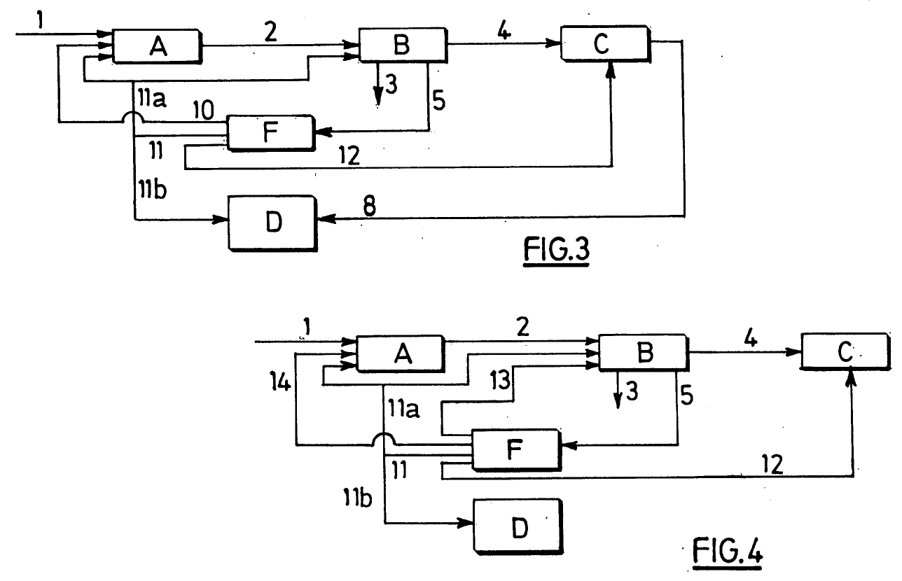

FIG. 3 is a flowchart of the method according to the invention.

The invention therefore relates to a method for converting gaseous hydrocarbons to liquid hydrocarbons in which the Fischer-Tropsch process is employed, said process producing liquid hydrocarbons and a waste gas comprising at least hydrogen, carbon monoxide, carbon dioxide and hydrocarbons with a maximum of 6 carbon atoms, and in which the waste gas is subjected to a separation method producing:
at least one gas stream comprising methane and for which the recovery rate of hydrogen and carbon monoxide is at least 60%,
at least one gas stream for which the carbon dioxide recovery rate is at least 40%, and
at least one supplementary gas stream mainly comprising hydrocarbons with at least 2 carbon atoms.

The invention relates to any type of method for converting gaseous hydrocarbons to liquid hydrocarbons using the Fischer-Tropsch process. In general, these gaseous hydrocarbons are produced by a reaction for producing a hydrocarbon synthesis gas (for example by partial oxidation using an oxidizing gas and steam). This synthesis gas comprises hydrogen and CO. It is normally produced by a unit for preparing a synthesis gas from natural gas or from an associated gas or from coal. According to the method of the invention, this synthesis gas is subjected to a Fischer-Tropsch reaction by contact with a catalyst promoting this reaction.

During the Fischer-Tropsch reaction, the hydrogen and CO are converted to hydrocarbon compounds of variable chain length by the following reaction:
CO+(1+m/2n)H₂→(1/n)CₙHₘ+H₂O

CO₂ is also produced during this reaction; for example by the following side reactions:
CO+H₂O→CO₂+H₂
2CO→CO₂+C

At the exit of the reactor using the Fischer-Tropsch process, the temperature of the products is generally lowered from a temperature of about 130° C. to a temperature of about 90 to 60° C., so that, on the one hand, a condensate is obtained, consisting mainly of water and liquid hydrocarbons with more than 4 carbon atoms, and, on the other hand, a waste gas comprising at least hydrogen, carbon monoxide, hydrocarbons with a maximum of 6 carbon atoms, carbon dioxide and generally also nitrogen.

The present invention relates to the treatment of this waste gas obtained. According to the method of the invention, this waste gas is subjected to a separation method producing:
at least one gas stream comprising methane and for which the recovery rate of hydrogen and carbon monoxide is at least 60%,
at least one gas stream for which the carbon dioxide recovery rate is at least 40%, and
at least one supplementary gas stream mainly comprising hydrocarbons with at least 2 carbon atoms. According to the invention, the recovery rate of a compound in one of the gas streams from the separation method corresponds to the volumetric or molar quantity of said compound present in the waste gas which is separated from said waste gas and which is produced in said gas stream from the separation method with respect to the total volumetric or molar quantity of this compound present in the waste gas. In the case of the gas stream for which the recovery rate of hydrogen and carbon monoxide is at least 60%, the condition of 60% recovery applies both to the CO compound with respect to the quantity of CO initially present in the waste gas and to the H₂ compound with respect to the quantity of H₂ initially present in the waste gas. According to the invention, “gas stream mainly comprising a compound” means a gas stream in which the concentration of this compound is higher than 50% by volume. According to the invention, the separation method used to treat the waste gas is advantageously a pressure swing adsorption (PSA) separation method. This PSA separation method is put into practice using a PSA separation unit used to obtain at least the following three main gas streams:
at least the first gas stream comprising methane and for which the recovery rate of hydrogen and carbon monoxide is at least 60%,
at least the second gas stream for which the carbon dioxide recovery rate is at least 40%, and
at least the third supplementary gas stream mainly comprising hydrocarbons with at least 2 carbon atoms. In general, for the first stream, the carbon monoxide recovery rate is lower than the hydrogen recovery rate (the recovery rate is about 60%-75% for carbon monoxide and about 75%-85% for hydrogen) while the methane recovery rate remains about 55%-65% and the CO₂ recovery rate remains below 1%. The CO₂ recovery rate in the second stream is higher than 40%, preferably higher than 50%. The third stream is a supplementary stream, and can therefore have a CO₂ recovery rate of at most 60%, preferably at most 50%. The second gas stream can comprise methane.

The separation method can also be used to produce at least one gas stream mainly comprising hydrogen. According to a first variant of the method according to the invention, the same PSA separation unit of the separation method used to treat the waste gas can also be used to produce at least one gas stream mainly comprising hydrogen. This stream can have a hydrogen concentration above 98% by volume. According to an alternative to this first variant of the method according to the invention, the separation method used to treat the waste gas can put into practice a second PSA separation unit intended to produce at least one gas stream mainly comprising hydrogen. This stream can have a hydrogen concentration above 98% by volume.

The waste gas can also comprise at least nitrogen and the waste gas separation method can produce at least one gas stream comprising at least nitrogen. In general, this gas stream comprising nitrogen corresponds to the gas stream mainly comprising hydrocarbons with at least 2 carbon atoms.

Preferably, each adsorber of the PSA separation unit is composed of at least three adsorbent beds:
the first bed being composed of alumina,
the second bed being composed of a silica gel, and
the third bed being composed of at least one adsorbent selected from either zeolites or carbon molecular sieves, with average pore sizes between 3.4 and 5 Å and preferably between 3.7 and 4.4 Å, or a titanium-silicate with average pore sizes between 3.4 and 5 Å, and preferably between 3.7 and 4.4 Å.

Depending on the different pressure cycles, the PSA separation method can be used to obtain in succession:
a high pressure gas stream comprising methane and for which the recovery rate of hydrogen and carbon monoxide is at least 60%, and
a gas stream for which the carbon dioxide recovery rate is at least 40%, and then
a supplementary gas stream mainly comprising hydrocarbons with at least 2 carbon atoms.

Alumina can be used to remove the water present in the waste gas and the hydrocarbon compounds with 5 or more carbon atoms. Silica gel can be used to adsorb the hydrocarbon compounds and particularly the hydrocarbon compounds with at least 3 carbon atoms. Preferably, the silica gel used has an alumina (Al₂O₃) content of less than 1% by weight. On the contrary, alumina and silica gel allow any H₂, CO and CH₄, and CO₂ and N₂ present in the waste gas to pass through. Zeolites or carbon molecular sieves with pore sizes as previously defined can be used to adsorb the carbon dioxide, and also partially the nitrogen. The choice of a titanium-silicate instead of the third zeolite bed or carbon molecular sieve bed also serves to retain the CO₂. The order of the three adsorbent beds is preferably the following, in the waste gas flow direction in the adsorber: first bed, then second bed, then third bed.

According to the first variant of the invention, each adsorber of the PSA separation unit can also comprise a fourth adsorbent bed in the waste gas flow direction in the adsorber; this fourth bed can be a zeolite or an activated charcoal if the third bed is a carbon molecular sieve. If the alternative to the first variant of the method according to the invention is put into practice, the adsorber of the second PSA separation unit producing at least one gas stream relatively pure in hydrogen (hydrogen concentration above 98% by volume) is composed of an adsorbent bed comprising at least one activated charcoal. In this case, at least a portion of the first stream from the first adsorption unit is introduced into this second adsorption unit.

Each adsorber of the PSA separation unit can also comprise a fourth or fifth bed comprising at least one titanium-silicate or one zeolite; this makes it possible to stop the nitrogen, at least partially. Preferably the titanium-silicate and zeolite have an average pore size of about 3.7 Å, or preferably between 3.5 Å and 3.9 Å; they are preferably exchanged with lithium, sodium, potassium or calcium, or are a combination of these elements. The structure of the zeolite is preferably selected from the following structures: LTA, CHA, AFT, AEI-AIPO18, KFI, AWW, SAS, PAU, RHO.

According to a first embodiment, downstream of the waste gas treatment, the gas stream from the separation method, comprising methane and for which the recovery rate of hydrogen and carbon monoxide is at least 60%, can be treated by a cryogenic unit in order to produce: either, according to a first version:
at least one stream essentially comprising hydrogen and carbon monoxide, and
at least one stream mainly comprising methane,
   or, according to a second version:
at least one stream essentially comprising hydrogen,
at least one stream mainly comprising carbon monoxide, and
at least one stream essentially comprising methane.

“Stream essentially comprising” a compound means a stream comprising at least 85% by volume of the compound, and preferably at least 95%. Thus, according to the first version, after decarbonation, and cooling of the gas stream comprising methane and for which the recovery rate of hydrogen and carbon monoxide is at least 60%, it is possible to use a column for separating the liquid phases condensed from the vapor phase, the vapor phase essentially consisting of hydrogen and CO, while the condensed phase mainly consists of methane. According to the second version, after decarbonation and cooling of the gas stream comprising methane to at least minus 150° C., for which the recovery rate of hydrogen and carbon monoxide is at least 60%, it is possible to use a methane scrubbing column to absorb the CO and to produce: at the top of the column in the vapor phase, a stream essentially comprising hydrogen, and at the bottom of the column, a condensed phase essentially containing methane and CO, which is sent to a CO/hydrocarbon distillation column to generate: at the top, a stream mainly comprising CO, and at the bottom, a stream essentially comprising methane.

According to a second embodiment, downstream of the waste gas treatment, the gas stream from the separation method, comprising methane and for which the recovery rate of hydrogen and carbon monoxide is at least 60%, can also be treated by a downstream PSA method in order to produce:
at least one stream essentially comprising hydrogen, and
at least one stream mainly comprising carbon monoxide and methane.

The various gases from the waste gas separation method can then be utilized in various parts of the GtL unit. Thus, at least a portion of the gas stream from the waste gas separation method, comprising methane and for which the recovery rate of hydrogen and carbon monoxide is at least 60%, can be used as reagent gas in a unit for preparing a synthesis gas comprising H₂ and CO, if any, and/or as reagent gas in the Fischer-Tropsch process. Similarly, at least a portion of the gas stream from the waste gas separation method, mainly comprising hydrocarbons with at least 2 carbon atoms, can be used as fuel and/or as reagent gas in the generation of synthesis gas. At least a portion of the gas stream from the waste gas separation method, mainly comprising hydrogen, can be used for hydrocracking processes, like the one used to treat liquid hydrocarbons with more than 4 carbon atoms and produced by the Fischer-Tropsch process. Finally, at least a portion of the gas stream from the waste gas separation method, for which the carbon dioxide recovery rate is at least 40%, can be used as reagent gas in a unit for preparing a synthesis gas comprising H₂ and CO, if any, or as reagent gas in the Fischer-Tropsch process. The latter case is useful when the Fischer-Tropsch catalyst produces CO₂ from CO; the reaction can then be equilibrated and the overproduction of CO₂ avoided. The removal of the methane from certain streams serves to prevent its accumulation during the recycling of these streams, particularly in the stream that is recycled to the Fischer-Tropsch process.

FIG. 1 shows a method of the prior art in a GtL type of plant. A raw gas (1) is treated in a unit for preparing a synthesis gas (A) to supply a synthesis gas (2) containing hydrogen and CO. This synthesis gas (2) is sent to a Fischer-Tropsch unit (B) where it is subjected to a Fischer-Tropsch reaction followed by condensation, for example in a settling drum.

The products from the Fischer-Tropsch unit are:
the condensate (3) from condensation which mainly comprises water. This condensate is removed from the GtL plant.
liquid hydrocarbon compounds (4) with more than 4 carbon atoms. These compounds are generally subjected to a treatment (C) for cutting their long chains and for obtaining chain lengths of at least 6 carbon atoms, for example, using hydrogen. The hydrocarbon compounds with a smaller number of carbon atoms (8) are used as fuel in an electricity generating unit (D).
a waste gas (5) comprising a mixture of H₂, CO, CO₂ and light hydrocarbons with a maximum of 6 carbon atoms, which can be either partially (6) reintroduced into the Fischer-Tropsch reactor, or partially (7) used as fuel in an electricity generating unit (D) or a steam production unit.

FIG. 2 shows the method put into practice in FIG. 1, but in which the waste gas (5) is treated by a CO₂ stripping unit (E). The C0₂ recovered in (9) is injected into the synthesis gas production unit (A).

FIG. 3 shows the method according to the invention. Unlike the methods of the prior art shown in FIGS. 1 and 2, the waste gas (5) comprising a mixture of H₂, CO, CO₂ and light hydrocarbons with a maximum of 6 carbon atoms, is treated at least partially (10) by a separation method (F) yielding:
a gas (11) mainly comprising hydrocarbons with at least 2 carbon atoms, which can partially (11a) be recycled to synthesis gas generation (A), or partially (11b) used as fuel in an electricity generating unit (D),
a gas (12) mainly comprising hydrogen. This gas (12) can be used during the treatment (C) to cut the chains of the liquid hydrocarbon compounds (4) from the Fischer-Tropsch process,
a gas (13) comprising hydrogen and carbon monoxide with a recovery rate of at least 60% and methane, which is recycled to the Fischer-Tropsch reactor (B), and
a gas (14) comprising CO₂ with a carbon dioxide recovery rate of at least 40%, which is introduced into the synthesis gas preparation unit (A)

1-19. (canceled)

20-43. (canceled)

44. A method which may be used for converting gaseous hydrocarbons to liquid hydrocarbons in which a Fischer-Tropsch process is employed, said method comprising:
a) producing liquid hydrocarbons and a waste gas, wherein said waste gas comprises:
1) hydrogen;
2) carbon dioxide; and
3) hydrocarbons with no more than 6 carbon atoms; and
b) separating said waste gas into at least three product streams, wherein said separation comprises the production of:
1) at least one gas stream comprising methane, wherein the recovery rate of hydrogen and carbon monoxide is at least about 60%;
2) at least one gas stream with a carbon dioxide recovery rate of at least about 40%; and
3) at least one supplementary gas stream, wherein said supplementary gas stream comprises hydrocarbons with at least 2 carbon atoms.

45. The method of claim 44, wherein said separation of said waste gas further comprises separation with a PSA separation unit having at least one adsorber.

46. The method of claim 45, further comprising producing at least one gas stream comprising hydrogen with said PSA separation unit.

47. The method of claim 45, wherein said separating said waste gas further comprises producing at least one gas stream comprising hydrogen with a second PSA separation unit.

48. The method of claim 45, wherein:
a) said waste gas further comprises nitrogen; and
b) said separation of said waste gas further comprises producing at least one gas stream comprising nitrogen.

49. The method of claim 45, wherein each adsorber of said PSA separation unit comprises:
a) a first bed comprising alumina;
b) a second bed comprising silica gel; and
c) a third bed comprising at least one adsorbent, wherein:
1) said adsorbent comprises at least one member selected from the group consisting of:
i) zeolite;
ii) carbon molecular sieves; and
iii) titanium silicate; and
2) said adsorbent has an average pore size between about 3.4 Å and about 5 Å.

50. The method of claim 49, wherein said average pore size is between about 3.7 Å and about 4.4 Å.

51. The method of claim 49, wherein said waste gas flows through said first bed, then through said second bed, and finally through said third bed.

52. The method of claim 49, wherein each said adsorber of said PSA separation unit further comprises a fourth adsorbent bed which is located, in said waste gas flow direction, after said third bed.

53. The method of claim 52, wherein:
a) said adsorbent of said third bed comprises carbon molecular sieves; and
b) said fourth bed comprises zeolite or an activated charcoal.

54. The method of claim 53, further comprising producing at least one gas stream comprising hydrogen with said PSA separation unit.

55. The method of claim 47, wherein an adsorber of said second PSA separation unit comprises an adsorbent bed comprising at least one activated charcoal.

56. The method of claim 49, wherein each adsorber of said PSA separation unit comprises a fourth or a fifth bed that comprises at least one member selected from the group consisting of:
a) titanium-silicate; and
b) zeolite.

57. The method of claim 56, wherein:
a) said waste gas comprises nitrogen; and
b) said separation of said waste gas further comprises producing at least one gas stream comprising nitrogen.

58. The method of claim 44, further comprising:
a) treating said at least one gas stream comprising methane with a cryogenic unit, wherein said treating occurs downstream of said waste gas separation;
b) producing at least one stream consisting essentially of hydrogen and carbon monoxide; and
c) producing at least one stream comprising methane.

59. The method of claim 44, further comprising:
a) treating said gas stream comprising methane with a cryogenic unit, wherein said treating occurs downstream of said waste gas separation;
b) producing at least one stream consisting essentially of hydrogen;
c) producing at least one stream comprising carbon monoxide; and
d) producing at least one stream consisting essentially of methane.

60. The method of claim 44, further comprising:
a) treating said gas stream comprising methane first with a PSA adsorber, wherein said treating occurs downstream of said waste gas separation;
b) producing at least one stream consisting essentially of hydrogen; and
c) producing at least one stream comprising carbon monoxide and methane.

61. The method of claim 44, further comprising synthesizing a gas comprising hydrogen and carbon monoxide from a reagent gas, wherein said reagent gas comprises at least a portion of said gas stream comprising methane.

62. The method of claim 44, wherein at least a portion of said gas stream comprising methane is used as a reagent gas in said Fischer-Tropsch process.

63. The method of claim 44, further comprising using at least a portion of said supplementary gas stream as fuel.

64. The method of claim 44, further comprising using at least a portion of said supplementary gas as a reagent gas for the generation of synthesis gas.

65. The method of claim 46, further comprising using at least a portion of said gas stream comprising hydrogen for hydrocracking.

66. The method of claim 47, further comprising using at least a portion of said gas stream comprising hydrogen for hydrocracking.

67. The method of claim 59, further comprising using at least a portion of said gas stream comprising hydrogen for hydrocracking.

68. The method of claim 60, further comprising using at least a portion of said gas stream comprising hydrogen for hydrocracking.

69. The method of claim 44, wherein at least a portion of said stream with a carbon dioxide recovery rate of at least about 40%, is used as a reagent gas for producing a synthesis gas which comprises hydrogen and carbon monoxide.

70. A method which may be used for converting gaseous hydrocarbons to liquid hydrocarbons in which a Fischer-Tropsch process is employed, wherein:
a) said method comprises:
1) producing liquid hydrocarbons and a waste gas, wherein said waste gas comprises:
i) hydrogen;
ii) carbon dioxide;
iii) hydrocarbons with no more than 6 carbon atoms; and
iv) nitrogen; and
2) separating said waste gas into at least three product streams, wherein said separation comprises the production of:
i) at least one gas stream comprising methane, wherein the recovery rate of hydrogen and carbon monoxide is at least about 60%;
ii) at least one gas stream with a carbon dioxide recovery rate of at least about 40%;
iii) at least one supplementary gas stream, wherein said supplementary gas stream comprises hydrocarbons with at least 2 carbon atoms; and
iv) at least one gas stream comprising nitrogen;
b) said separating said waste gas further comprises separation with a PSA separation unit having at least one adsorber;
c) each said adsorber of said PSA separation unit comprises:
1) a first bed comprising alumina;
2) a second bed comprising silica gel;
3) a third bed comprising at least one adsorbent, wherein:
i) said adsorbent comprises at least one member selected from the group consisting of:
aa) zeolite;
bb) carbon molecular sieves; and
cc) titanium silicate; and
ii) said adsorbent has an average pore sized between about 3.4 Å and about 5 Å; and
4) a fourth bed comprising at least one member selected from the group consisting of:
i) titanium-silicate; and
ii) zeolite; and
d) at least one gas stream comprising hydrogen is produced by said PSA separation unit.

71. A method which may be used for converting gaseous hydrocarbons to liquid hydrocarbons in which a Fischer-Tropsch process is employed, said method comprising:
a) producing liquid hydrocarbons and a waste gas, wherein said waste gas comprises:
1) hydrogen;
2) carbon dioxide; and
3) hydrocarbons with no more than 6 carbon atoms;
b) separating said waste gas into at least three product streams, with a PSA separation unit, wherein said separation comprises the production of:
1) at least one gas stream comprising methane, wherein the recovery rate of hydrogen and carbon monoxide is at least about 60%;
2) at least one gas stream with a carbon dioxide recovery rate of at least about 40%; and
3) at least one supplementary gas stream, wherein said supplementary gas stream comprises hydrocarbons with at least 2 carbon atoms;
c) treating said gas stream comprising methane with a cryogenic unit, wherein said treating comprises:
1) producing at least one stream consisting essentially of hydrogen;
2) producing at least one stream comprising carbon monoxide; and
3) producing at least one stream consisting essentially of methane; and
d) hydrocracking at least a portion of said stream consisting essentially of hydrogen.
